(19) United States
(12) Patent Application Publication
(10) Pub. No.: US 2026/0097092 A1
(43) Pub. Date: Apr. 9, 2026

(54) PHARMACEUTICAL COMPOSITION CONTAINING MITOCHONDRIA-TARGETING COMPOUND AS ACTIVE INGREDIENT FOR TREATING MACULAR DEGENERATION

(71) Applicants: ULSAN NATIONAL INSTITUTE OF SCIENCE AND TECHNOLOGY, Ulsan (KR); KONKUK UNIVERSITY GLOCAL INDUSTRY-ACADEMIC COLLABORATION FOUNDATION, Chungju-si Chungcheongbuk-do (KR)

(72) Inventors: Ja Hyoung RYU, Ulsan (KR); Sang Pil KIM, Ulsan (KR); Do Hyun KIM, Ulsan (KR); Hye Won CHUNG, Seoul (KR); Jae Byoung CHAE, Seongnam-si Gyeonggi-do (KR)

(73) Assignees: ULSAN NATIONAL INSTITUTE OF SCIENCE AND TECHNOLOGY, Ulsan (KR); KONKUK UNIVERSITY GLOCAL INDUSTRY-ACADEMIC COLLABORATION FOUNDATION, Chungju-si Chungcheongbuk-do (KR)

(21) Appl. No.: 19/113,607

(22) PCT Filed: Jan. 11, 2023
(86) PCT No.: PCT/KR2023/000520

§ 371 Date: Apr. 16, 2025

(30) Foreign Application Priority Data
Sep. 21, 2022 (KR) 10-2022-0119426

(51) Int. Cl. A61K 38/08; A61P 27/02

(52) U.S. Cl. CPC A61K 38/08; A61P 27/02

(57) ABSTRACT

The present disclosure relates to a pharmaceutical composition for treating retinal diseases, including a mitochondria-targeting compound as an active ingredient, wherein according to one aspect, the compound or a pharmaceutically acceptable salt thereof, and a pharmaceutical composition comprising the same may specifically induce apoptosis of senescent cells, and thus be usefully applied for the effective prevention or treatment of aging-related diseases.

### TECHNICAL FIELD

The present disclosure relates to a pharmaceutical composition for treating retinal diseases, including a mitochondria-targeting compound as an active ingredient.

### BACKGROUND ART

Normal vision occurs when light enters the front of the eye and is focused by the lens onto extremely sensitive, delicate cells called photoreceptors, which line the interior back portion of the eye. Photoreceptors, together with other neurons, constitute the retina of the eye. The macula is a part of the retina located near the center of the back of the eye, which includes the fovea, responsible for sharp central vision. The macula is the neural tissue situated in the central portion of the inner retina, where the majority of photoreceptors responsive to light stimuli are concentrated, and since the center of the macula is also where an object's image is formed, it plays an extremely important role in central vision.

The retina can be affected by various conditions, such as retinitis, macular degeneration, diabetic retinopathy, macular edema, retinal injury, glaucoma, optic neuropathy, retinal vascular diseases, and ocular vascular diseases triggered by diverse causes. In particular, the ophthalmological disease that causes visual impairment due to degeneration in the macular region (retinal pigment epithelium, Bruch's membrane, and choroidal capillary complex) caused by various complex factors is called age-related macular degeneration (hereinafter, macular degeneration, AMD). Macular degeneration is known as one of the three major causes of blindness along with glaucoma and diabetic retinopathy. Macular degeneration can affect central vision, causing blurriness in the center of the visual field, central scotomas, metamorphopsia (perception of distorted shapes), or localized vision loss. As a result, it may hinder precise activities such as reading or driving, and in severe cases can lead to blindness.

Aging is considered the single most prominent cause of macular degeneration, although factors such as family history, ethnicity, and smoking are also known to be somewhat correlated. Macular degeneration has long been the most common cause of blindness among the elderly in Western societies. In the United States, it already ranks as the number-one cause of blindness in older adults, and in Korea, the number of patients has been rapidly increasing in step with the accelerated aging of the population. This phenomenon is thought to stem from both the adoption of a Western diet and the increased exposure to UV rays resulting from ozone-layer depletion. While macular degeneration traditionally appeared mainly in the elderly, the age of onset in Korea has recently shifted from the elderly in their 60s to the middle-aged in their 40s and 50s, and the incidence rate in the middle-aged population has also risen sharply over the past few years.

Macular degeneration can be broadly classified into two types: dry and wet. Approximately 90% of macular degeneration patients have the dry form. Age-related macular degeneration (AMD) frequently begins in this dry state. In dry macular degeneration, early symptoms are often absent, making self-diagnosis difficult. Over time, the disease gradually advances and eventually progresses to atrophic macular degeneration (geographic atrophy), resulting in a loss of central vision. Since there is currently no established treatment for dry macular degeneration, the need for prevention is all the more critical.

### DISCLOSURE OF INVENTION

### Technical Problem

An aspect is to provide a pharmaceutical composition for preventing or treating a retinal disease, the composition including, as an active ingredient, a compound represented by Formula 1 below or a pharmaceutically acceptable salt thereof:

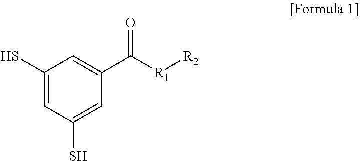

Here, R₁ is a mitochondria-targeting peptide and R₂ is a senescent cell-targeting peptide.

Another aspect is to provide a health functional food for preventing or alleviating a retinal disease, the health functional food including, as an active ingredient, a compound represented by Formula 1 above or a pharmaceutically acceptable salt thereof.

Another aspect is to provide a method of preventing or treating a retinal disease, the method including administering a compound represented by Formula 1 below or a pharmaceutically acceptable salt thereof, to a subject in need thereof:

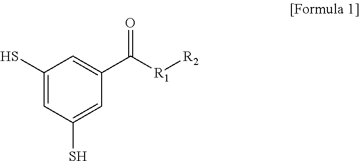

In Formula 1, R₁ is a mitochondria-targeting peptide and R₂ is a senescent cell-targeting peptide.

Another aspect is to provide use of a compound represented by Formula 1 below or a pharmaceutically acceptable salt thereof in the manufacture of a drug for preventing or treating a retinal disease:

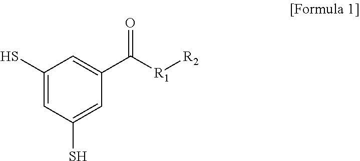

a.

In Formula 1, R₁ is a mitochondria-targeting peptide and R₂ is a senescent cell-targeting peptide.

### Solution of Problem

One aspect provides a pharmaceutical composition for preventing or treating a retinal disease, the composition including, as an active ingredient, a compound represented by Formula 1 below or a pharmaceutically acceptable salt thereof:

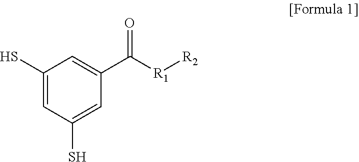

Here, R₁ is a mitochondria-targeting peptide and R₂ is a senescent cell-targeting peptide.

Another aspect provides a method of preventing or treating a retinal disease, the method including administering a compound represented by Formula 1 below or a pharmaceutically acceptable salt thereof to a subject in need thereof:

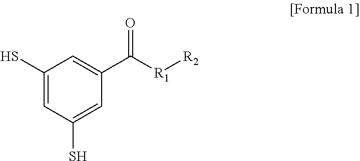

In Formula 1, R₁ is a mitochondria-targeting peptide, and R₂ is a senescent cell-targeting peptide.

The mitochondria-targeting peptide refers to one that has the function of targeting the compound to mitochondria within cells, and may include an MTS (mitochondria targeting sequence). Specifically, the mitochondria-targeting peptide may be Leu-Leu-Arg-Ala-Ala-Leu-Arg-Lys-Ala-Ala-Leu (LLRAALRKAAL), Met-Leu-Arg-Ala-Ala-Leu-Ser-Thr-Ala-Arg-Arg-Gly-Pro-Arg-Leu-Ser-Arg-Leu-Leu (MLRAALSTARRGPRLSRLL), Met-Leu-Ser-Leu-Arg-Gln-Ser-Ile-Arg-Phe-Phe-Lys (MLSLRQSIRFFK), Leu-Ser-Arg-Thr-Arg-Ala-Ala-Ala-Pro-Asn-Ser-Arg-Ile-Phe-Thr-Arg (LSRTRAAAPNSRIFTR), Met-Ile-Ala-Ser-His-Leu-Leu-Ala-Tyr-Phe-Phe-Thr-Glu-Leu-Asn (MIASHLLAYFFTELN), Lys-Leu-Ala-Lys-Leu-Ala-Lys (KLAKLAK), Lys-Leu-Ala-Lys-Arg-Gly-Asp (KLAKRGD), or Lys-Leu-Ala-Lys-Leu-Ala-Lys-Arg-Gly-Asp (KLAKLAKRGD). As long as the peptide includes a KLAK or RGD sequence, it is not limited to the aforementioned examples. In addition, each of the listed amino acids may include a modified amino acid.

The senescent cell-targeting peptide may be Arg-Gly-Asp (RGD).

The senescent cell-targeting peptide may selectively recognize and bind integrin αvβ3 on the mitochondrial membrane.

The peptide may be connected from the N-terminus to the C-terminus by peptide bonds in the order of the amino acids, and the hydroxyl group on the carboxyl terminus may be substituted with one selected from the group consisting of an amine group, an alcohol group, an ether group, and an acetyl group. Alternatively, no particular chemical group may be bonded, or another chemical group may be chemically attached; for example, an amine group could be introduced.

In the present disclosure, the compound may be targeted to the mitochondria inside cells due to the mitochondria-targeting peptide, and the compound can pass through the mitochondrial double membrane and enter the interior of the mitochondria.

In the present disclosure, the compound can enter the mitochondria due to the aforementioned mitochondria-targeting peptide. Once inside the mitochondria, the compound can selectively form macromolecules within the mitochondria of senescent cells, thereby inducing apoptosis.

In an embodiment, the compound represented by Formula 1 may be Mito-K1 or Mito-K2.

The —SH group of Formula 1 may be capable of undergoing oxidation to form a bond with a plurality of other molecules of the compound. Specifically, the —SH group may be oxidized to form a disulfide bond (—S—S—) with another molecule of the compound, and through this bond, the compound may bind with other molecules of the compound to form a macromolecule.

In an embodiment, the macromolecule may result from oligomerization, induced by disulfide bond formation between the compound and other molecules of the compound.

The aforementioned oxidation may be caused by reactive oxygen species (ROS).

In the present disclosure, the compound is oxidized by ROS, which are overexpressed in senescent cells compared to normal cells, leading to the formation of macromolecules within the mitochondria of senescent cells, thereby disrupting the integrity of the mitochondrial membrane and selectively inducing apoptosis of the senescent cells.

In an embodiment, the retinal disease may be one selected from the group consisting of macular degeneration, diabetic retinopathy, choroidal neovascularization, and retinal edema.

Throughout this specification, the term “retinal disease” refers to a disease resulting from damage to the retina due to reasons such as aging, genetic abnormalities, or disease, and may be any one selected from the group consisting of macular degeneration, diabetic retinopathy, choroidal neovascularization, and retinal edema.

Macular degeneration is a disease in which macular function deteriorates with aging, leading to decreased or lost vision, and is also called age-related macular degeneration. Macular degeneration is classified into dry macular degeneration and wet macular degeneration, and is a major cause of vision loss in old age.

Diabetic retinopathy is a complication that occurs in the retina of the eye due to peripheral circulatory disorders caused by diabetes, and symptoms of decreased vision occur as the macula is involved.

Choroidal neovascularization is a disease in which abnormal blood vessels develop from the choroid and invade the retina, causing bleeding, edema, and other problems, leading to visual impairment.

Retinal edema occurs when degeneration or abnormalities occur in microvessels such as capillaries in the retina or macula, resulting in swelling of the retina and decreased vision.

In an embodiment, the macular degeneration may be one or more selected from the group consisting of age-related macular degeneration (AMD), wet macular degeneration, and dry macular degeneration. Dry macular degeneration refers to the condition where lesions such as drusen (accumulation of waste products in the macula) or atrophy of the retinal pigment epithelium occur in the retina, and wet macular degeneration refers to a disease caused by choroidal neovascularization growing under the retina. Administration of the compound of Formula 1 according to the present disclosure to an aged retina may be effective for macular degeneration. In an embodiment, it was confirmed that administration of Mito-K2 or Mito-K1 results in oligomerization via intermolecular disulfide bond formation within the mitochondria, disrupting the integrity of the mitochondrial membrane and selectively inducing apoptosis of senescent cells.

The term “therapeutic agent” or “pharmaceutical composition” as used herein refers to a molecule or compound that imparts one or more beneficial effects upon administration to a subject. Such beneficial effects may include enabling diagnostic decisions; alleviating a disease, symptom, disorder, or pathological condition; reducing or preventing the onset of a disease, symptom, disorder, or pathological condition; and, in general, addressing the disease, symptom, disorder, or pathological condition.

The pharmaceutical composition of the present disclosure may take any form suitable for the intended route of administration. In the present disclosure, “administration” of a pharmaceutical composition means introducing a given substance into a patient by any appropriate method, and the pharmaceutical composition can be delivered through any common route as long as the drug can reach the target tissue. For example, such administration routes may include ocular local administration (e.g., peribulbar (e.g., sub-Tenon's), subconjunctival, intraocular, intravitreal, anterior chamber, subretinal, suprachoroidal, and retrobulbar injections), intraperitoneal administration, intravenous administration, intramuscular administration, subcutaneous administration, intradermal administration, oral administration, topical administration, nasal administration, pulmonary administration, rectal administration, and so on, but are not limited thereto. Furthermore, the pharmaceutical composition of the present disclosure may be administered by any device that allows the active ingredient to reach the target cells. The route of administration of the pharmaceutical composition of the present disclosure is preferably determined according to the type of disease to which it is applied.

The pharmaceutical composition provided herein may be formulated according to conventional methods into oral dosage forms such as powders, granules, tablets, capsules, suspensions, emulsions, syrups, and aerosols, or into parenteral dosage forms such as suspensions, emulsions, freeze-dried preparations, external preparations, suppositories, sterile injectable solutions, and implantable formulations.

The pharmaceutical composition may further comprise pharmaceutically acceptable excipients, in addition to the active ingredient (i.e., the compound or a pharmaceutically acceptable salt thereof), that are suitable for use in formulation.

Excipients that may be used in formulating the pharmaceutical composition of the present disclosure may include one or more selected from the group consisting of carriers, vehicles, diluents, and solvents (e.g., monohydric alcohols such as ethanol and isopropanol, and polyhydric alcohols such as glycerol, and edible oils such as soybean oil, coconut oil, olive oil, safflower oil, and cottonseed oil, oily esters such as ethyl oleate and isopropyl myristate); binders, adjuvants, solubilizers, thickeners, stabilizers, disintegrants, glidants, lubricants, buffering agents, emulsifiers, wetting agents, suspending agents, sweeteners, colorants, flavoring agents, coating agents, preservatives, antioxidants, and processing aids; and drug delivery modifiers and enhancers (e.g., calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidone, low melting point waxes, and ion-exchange resins), without being limited thereto.

The pharmaceutically acceptable carriers included in the pharmaceutical composition of the present disclosure are those commonly used in formulation, and include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil. The pharmaceutical composition of the present disclosure may further comprise, in addition to the above-mentioned ingredients, lubricants, wetting agents, sweeteners, flavoring agents, emulsifiers, suspending agents, and preservatives. Suitable pharmaceutically acceptable carriers and formulations are described in detail in Remington's Pharmaceutical Sciences (19th ed., 1995).

The pharmaceutical composition can be formulated in various oral dosage forms. For example, the pharmaceutical composition can be in any oral dosage form, such as tablets, pills, hard or soft capsules, solutions, suspensions, emulsions, syrups, granules, and elixirs. In addition to the active ingredient, such oral dosage forms may include pharmaceutically acceptable carriers, for instance, lactose, dextrose, sucrose, mannitol, sorbitol, cellulose, and/or glycine as diluents, or silica, talc, stearic acid or its magnesium or calcium salts, and/or polyethylene glycol as lubricants, depending on the standard composition required for each formulation.

If the oral dosage form is a tablet, it may include a binder such as magnesium aluminum silicate, starch paste, gelatin, tragacanth, methylcellulose, sodium carboxymethylcellulose, and/or polyvinylpyrrolidone. In some cases, such a tablet may also contain a disintegrant such as starch, agar, alginic acid, or a sodium salt thereof, as well as effervescent mixtures and/or absorbents, colorants, flavoring agents, or sweeteners.

Furthermore, the pharmaceutical composition may also be formulated in the form of a parenteral dosage form, which can be administered by a parenteral route such as subcutaneous injection, intravenous injection, intramuscular injection, or intrathoracic injection. In this case, to formulate into the parenteral dosage form, the pharmaceutical composition may be prepared by mixing the active ingredient with a stabilizer or buffering agent in water to prepare a solution or suspension. This solution or suspension may be prepared in unit dosage forms such as ampoules or vials.

Furthermore, the pharmaceutical composition may be sterilized or may further include adjuvants such as preservatives, stabilizers, hydrating agents, or emulsifiers, salts for regulating osmotic pressure, and/or buffers, and may also include other therapeutically useful substances, and may be formulated by conventional methods such as mixing, granulation, or coating.

The content of the compound or a pharmaceutically acceptable salt thereof in the pharmaceutical composition may be appropriately adjusted depending on the intended use of the pharmaceutical composition, the form of the formulation, etc., and may be, for example, 0.001 wt % to 99 wt %, 0.001 wt % to 90 wt %, 0.001 wt % to 50 wt %, 0.01 wt % to 50 wt %, 0.1 wt % to 50 wt %, or 1 wt % to 50 wt %, based on the total weight of the pharmaceutical composition, but is not limited thereto.

Furthermore, a therapeutically effective amount of the compound or a pharmaceutically acceptable salt thereof included in the pharmaceutical composition of the present disclosure refers to the amount required to produce the intended therapeutic effect upon administration. Therefore, the therapeutically effective amount may be adjusted depending on the type of disease of the patient, the severity of the disease, the type of active ingredient administered, the type of formulation, the age, sex, weight, health status, and diet of the patient, and the time and method of administration of the drug. For example, for mammals, including humans, such a pharmaceutically effective amount may be administered at 0.01 mg/kg to 500 mg/kg (body weight) per day. The pharmaceutically effective amount may be selected to achieve the desired effect, such as the treatment and/or prevention of an aging-related disease. The pharmaceutically effective amount may be administered once a day or in two or more divided doses via oral or parenteral routes (e.g., ocular injection, intravenous injection, intramuscular injection, etc.).

By administering the pharmaceutical composition in an amount effective for preventing or treating a retinal disease, specifically macular degeneration, to a subject, the retinal disease (specifically, macular degeneration) may be treated in the subject.

The subject may be a mammal. The mammal may be a human, dog, cat, cow, goat, or pig.

As used herein, the terms “treatment,” “treating,” “alleviating,” or “ameliorating” are used interchangeably. These terms refer to a method of obtaining a beneficial or desired outcome that may include, but is not limited to, a therapeutic benefit and/or a preventive benefit. Therapeutic benefit means any therapeutically significant improvement in, or effect on, one or more diseases, disorders, or symptoms under treatment. For prophylactic benefit, the composition may be administered to a subject at risk of developing a particular disease, disorder, or symptom, or to a subject reporting one or more physiological symptoms of a disease, even though the disease, disorder, or symptom may not yet be apparent.

In the present specification, the term “effective amount” or “therapeutically effective amount” refers to the amount of an agent sufficient to achieve a beneficial or desired outcome. A therapeutically effective amount may vary depending on one or more factors, such as the subject being treated and the pathological condition, the body weight and age of the subject, the severity of the condition, and the mode of administration, and may be readily determined by those skilled in the art. Furthermore, the term applies to a dosage sufficient to provide an image for detection by any of the imaging methods described herein. The specific dosage may vary depending on one or more of the particular agent selected, the dosage regimen followed, whether it is administered in combination with other compounds, the timing of administration, the tissue being imaged, and the physical delivery system delivering the dosage.

Another aspect provides a health functional food for preventing or alleviating a retinal disease, the health functional food including, as an active ingredient, a compound represented by Formula 1 below or a pharmaceutically acceptable salt thereof:

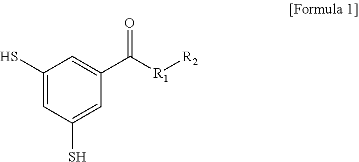

Here, R₁ is a mitochondria-targeting peptide and R₂ is a senescent cell-targeting peptide.

In an embodiment, the compound may be Mito-K1 or Mito-K2.

In an embodiment, the retinal disease may be one selected from the group consisting of macular degeneration, diabetic retinopathy, choroidal neovascularization, and retinal edema.

As used herein, the term “health functional food” refers to a food manufactured and processed using raw materials or ingredients having functionality beneficial to the human body, in accordance with Act No. 6727 on Health Functional Foods, and is intended to be ingested for the purpose of obtaining beneficial effects for health purposes, such as regulating nutrients or physiological functions related to the structure and function of the human body.

When the composition of the present disclosure is used as a food additive, the composition may be added directly or used in combination with other foods or ingredients, and may be used appropriately according to conventional methods. The amount of the active ingredient to be mixed may be suitably determined depending on the intended use. The food additive refers to a substance that is usually intentionally added to food in small amounts for the purpose of improving appearance, flavor, texture, or shelf life, and is intended to improve the quality, preservation, or palatability of food, as well as to enhance nutritional value and the substantial value of the food. This may be a substance used in the manufacture, processing, or preservation of food by addition, mixing, infiltration, or other methods, as defined in Article 2(2) of the Food Sanitation Act.

There is no particular limitation on the type of food of the present disclosure. Examples of foods to which the composition of the present disclosure can be added include meat products, sausages, breads, chocolates, candies, snacks, confectioneries, ramen, other noodle products, gums, dairy products including ice cream, soups, beverages, teas, health drinks, alcoholic drinks, vitamin complexes, and may include all conventionally known foods, as well as foods used as animal feed.

The health functional food of the present disclosure may contain conventional food additives, and the suitability as a food additive is determined according to the specifications and standards for the relevant item, unless otherwise specified, in accordance with the General Provisions and General Test Methods of the Food Additive Code approved by the Ministry of Food and Drug Safety.

Furthermore, the food composition of the present disclosure may contain various nutrients, vitamins, electrolytes, flavor enhancers, colorants, pectic acid or salts thereof, alginic acid or salts thereof, organic acids, protective colloid thickeners, pH regulators, stabilizers, preservatives, glycerin, alcohol, carbonating agents used in carbonated beverages, and so forth. The food composition may also contain fruit pulp for the production of natural fruit juices, fruit juice beverages, and vegetable beverages.

Moreover, the food may be formulated into tablets, granules, powders, capsules, liquid solutions, or pills according to known manufacturing methods. Other than incorporating the compound of the present disclosure as an active ingredient, there is no particular limitation on other components, and various conventional flavoring agents or natural carbohydrates, among others, may be included as additional components.

For example, a health functional food in the form of a tablet may be prepared by granulating, in a conventional manner, a mixture of the compound of Formula 1 of the present disclosure (as the active ingredient) with excipients, binders, disintegrants, and other additives, followed by compression molding with a lubricant or by directly compressing the mixture. Also, the health functional food in tablet form may, if necessary, contain corrigents and the like.

Among capsule-type health functional foods, a hard capsule formulation may be manufactured by filling conventional hard capsules with a mixture of the active ingredient of the present disclosure, the compound of Formula 1, and additives such as excipients, while soft capsules may be manufactured by filling a capsule base such as gelatin with a mixture of the compound of Formula 1 and additives such as excipients. The soft capsules may contain, if necessary, plasticizers such as glycerin or sorbitol, colorants, preservatives, and the like.

A health functional food in the form of a pill can be prepared by forming a mixture of the compound of Formula 1 of the present disclosure (as the active ingredient) with excipients, binders, disintegrants, and so on, using methods known in the art. If desired, the pill may be sugar-coated with sucrose or other coating agents, or the surface may be coated with substances such as starch or talc.

A health functional food in the form of granules may be produced, according to known methods, by granulating a mixture of the compound of Formula 1 of the present disclosure (as the active ingredient) with excipients, binders, disintegrants, etc. If necessary, the granules may also contain flavorants, corrigents, or the like.

### Advantageous Effects of Invention

According to an aspect, a compound, or a pharmaceutically acceptable salt thereof, and a pharmaceutical composition including the same, selectively induce apoptosis in senescent cells and are therefore useful for the effective prevention or treatment of diseases associated with aging.

### BRIEF DESCRIPTION OF DRAWINGS

FIG. 1 is a graph analyzing self-assembled polymer formation of Mito-K1 and Mito-K2 in a mitochondria-mimicking environment using MALDI-TOF mass spectrometry spectra.

FIG. 2 is a graph analyzing self-assembled polymer formation of Mito-K1 and Mito-K2 in an environment without hydrogen peroxide using MALDI-TOF mass spectrometry spectra.

FIG. 3 shows TEM images of Mito-K1 and Mito-K2 in a mitochondria-mimicking environment.

FIG. 4 is a graph showing the results of analyzing the size distribution of nanostructures formed via oligomerization of Mito-K1, Mito-K2, and Mito-K2-OH, using dynamic light scattering (DLS).

FIG. 5 is a SAXS graph analyzing the formation of nanostructures formed via oligomerization of Mito-K1, Mito-K2, and Mito-K2-OH.

FIG. 6 is a graph showing the formation and distribution of secondary structures of Mito-K1 and Mito-K2 at varying concentrations, using circular dichroism (CD) spectra.

FIG. 7 is an immunofluorescence image showing the mitochondrial targeting of Mito-K1, Mito-K2-FITC, and Mito-K2-OH.

FIG. 8 is a graph showing mitochondrial fluorescence levels in ARPE-19 cells and senescence-induced ARPE-19 cells at different Mito-K2-FITC concentrations.

FIG. 9 is a graph showing the analysis of self-assembled polymer formation of Mito-K1, Mito-K2, and Mito-K2-OH in Dox-induced senescent ARPE-19 cells.

FIG. 10 is a TEM image showing mitochondrial damage observed in Dox-induced senescent ARPE-19 cells following treatment with Mito-K2.

FIG. 11 is a schematic diagram and graph showing the release of internal fluorescence staining following treatment with Mito-K1, Mito-K2, and Mito-K2-OH in a mitochondria-mimicking membrane structure in Dox-induced senescent ARPE-19 cells.

FIG. 12 is an image and graph showing the change in mitochondrial membrane potential following treatment with Mito-K1, Mito-K2, and Mito-K2-OH in Dox-induced senescent ARPE-19 cells.

FIG. 13 is an image and graph showing the change in mitochondrial ROS formation following treatment with Mito-K1, Mito-K2, and Mito-K2-OH in Dox-induced senescent ARPE-19 cells.

FIG. 14 is an image showing the morphological changes in mitochondria following treatment with Mito-K2 in Dox-induced senescent ARPE-19 cells.

FIG. 15 is a graph analyzing the change in mitochondrial ATP levels following treatment with Mito-K1, Mito-K2, and Mito-K2-OH in Dox-induced senescent ARPE-19 cells.

FIG. 16 is a graph showing the cytotoxicity following treatment with Mito-K1, Mito-K2, and Mito-K2-OH in ARPE cells and Dox-induced senescent ARPE-19 cells.

FIG. 17 is a graph showing the induction of apoptosis following treatment with Mito-K2 in Dox-induced senescent ARPE-19 cells.

FIG. 18 is a graph showing changes in apoptosis-related factors (p21, p53, cCasp 9) following treatment with Mito-K1 and Mito-K2 in Dox-induced senescent ARPE-19 cells.

FIG. 19 is an image showing a reduction in the area of senescence following administration of Mito-K2 to Dox-induced senescent cells in mouse retinal tissue.

FIG. 20 is an image and graph showing a reduction in mitochondrial ROS generation following administration of Mito-K2 to Dox-induced senescent cells in mouse retinal tissue.

FIG. 21 is an image showing a decrease in the expression of apoptosis-related factors (p21, p53, ApoE) following administration of Mito-K2 to Dox-induced senescent cells in retinal tissue.

FIG. 22 is a graph showing the changes in mRNA levels of senescence markers and SASP factors following administration of Mito-K2 to Dox-induced senescent cells in retinal tissue.

FIG. 23 is an image showing an increase in cell proliferative activity in non-senescence-induced cells following administration of Mito-K2 to Dox-induced senescent cells in retinal tissue.

FIG. 24 is an image showing a reduction in the area of senescence following administration of Mito-K2 to Alu-induced senescent cells in retinal tissue.

FIG. 25 is an image and graph showing a reduction in mitochondrial ROS generation following administration of Mito-K2 to Alu-induced senescent cells in retinal tissue.

FIG. 26 is an image and graph showing the recovery of TOM20 expression levels following administration of Mito-K2 to Alu-induced senescent cells in retinal tissue.

FIG. 27 is a graph showing an increase in the mitochondrial oxygen consumption rate following administration of Mito-K2 to Alu-induced senescent cells in retinal tissue.

FIG. 28 is a UMAP plot showing the expression levels of genes in RPE cells from the control group, Alu-induced senescence group, and Mito-K2-treated group, classified into seven subgroups.

FIG. 29 is a graph showing the results of gene ontology analysis for senescence-related genes and mitochondrial genes in the control group and Alu group.

FIG. 30 is a graph showing the results of gene ontology analysis for some of the senescence-related genes in the Alu group and Mito-K2 group.

FIG. 31 is a graph showing the results of gene ontology analysis for genes of RPE cells, rod cells, and cone cells in the Alu group and Mito-K2 group.

FIG. 32 is a graph showing the results of measuring electroretinograms of RPE cells in the control group, Alu group, and Mito-K2 group.

### BEST MODE FOR CARRYING OUT THE INVENTION

One aspect provides a pharmaceutical composition for preventing or treating a retinal disease, the composition including, as an active ingredient, a compound represented by Formula 1 below or a pharmaceutically acceptable salt thereof:

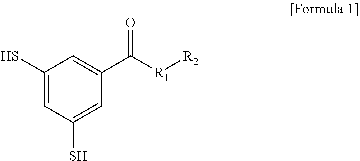

In Formula 1, R₁ is a mitochondria-targeting peptide, and R₂ is a senescent cell-targeting peptide.

In an embodiment, the mitochondria-targeting peptide may be Lys-Leu-Ala-Lys (KLAK) or Lys-Leu-Ala-Lys-Leu-Ala-Lys (KLAKLAK).

In an embodiment, the senescent cell-targeting peptide may be Arg-Gly-Asp (RGD).

In an embodiment, the compound may be Mito-K1 or Mito-K2.

In an embodiment, the compound in the composition may be capable of undergoing oxidation to form disulfide bonds with other molecules of the compound.

In an embodiment, the compound in the composition may form a self-assembled polymer via oligomerization within mitochondria of senescent cells.

In an embodiment, the compound in the composition may induce apoptosis in mitochondria of senescent cells.

In an embodiment, the retinal disease may be one selected from the group consisting of macular degeneration, diabetic retinopathy, choroidal neovascularization, and retinal edema.

In an embodiment, the macular degeneration may be one or more selected from the group consisting of age-related macular degeneration (AMD), wet macular degeneration, and dry macular degeneration.

Another aspect provides a health functional food for preventing or alleviating a retinal disease, the health functional food comprising, as an active ingredient, a compound represented by Formula 1 or a pharmaceutically acceptable salt thereof:

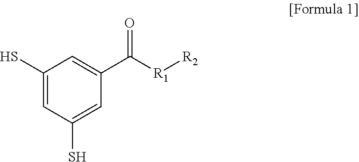

In Formula 1, R₁ is a mitochondria-targeting peptide and R₂ is a senescent cell-targeting peptide.

In an embodiment, the compound may be Mito-K1 or Mito-K2.

In an embodiment, the retinal disease may be one selected from the group consisting of macular degeneration, diabetic retinopathy, choroidal neovascularization, and retinal edema.

Another aspect provides a method of preventing or treating a retinal disease, the method including administering a compound represented by Formula 1 or a pharmaceutically acceptable salt thereof, to a subject in need thereof:

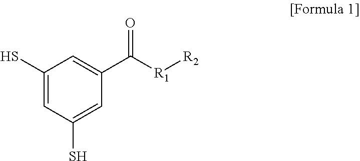

In Formula 1, R₁ is a mitochondria-targeting peptide and R₂ is a senescent cel-targeting peptide.

One aspect provides use of a compound represented by Formula 1, or a pharmaceutically acceptable salt thereof, in the manufacture of a composition for preventing or treating a retinal disease:

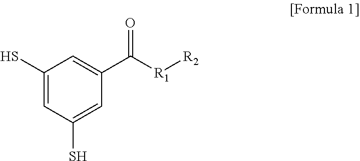

In Formula 1, R₁ is a mitochondria-targeting peptide and R₂ is a senescent cell-targeting peptide.

### MODE FOR THE INVENTION

The present disclosure will now be described in greater detail through the following examples. However, these examples are provided for illustrative purposes only and are not intended to limit the scope of the present disclosure.

### Experimental Example 1. Confirmation of Peptide Oligomerization and Formation of Stable Helical Self-Assembly

Mito-K1 and Mito-K2, which are mitochondrial-targeting compounds having specificity for mitochondria, were prepared by solid-phase peptide synthesis (Solid Phase Peptide Synthesis). These Mito-K1 and Mito-K2 were designed by varying the repeat number of KLAK, a sequence known to interact with artificial nanostructures and mitochondrial membranes. In addition, to confirm the functionality of disulfide bonds, Mito-K2-OH, which has a dihydroxy group instead of a dithiol group, was designed as a control molecule.

### 1.1. Confirmation of Peptide Oligomer Formation Under Oxidative Conditions

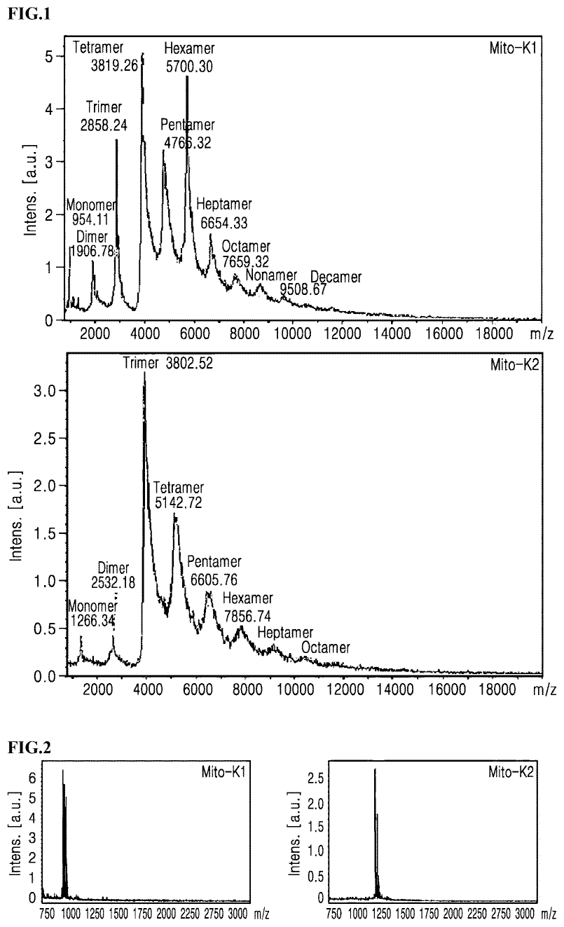

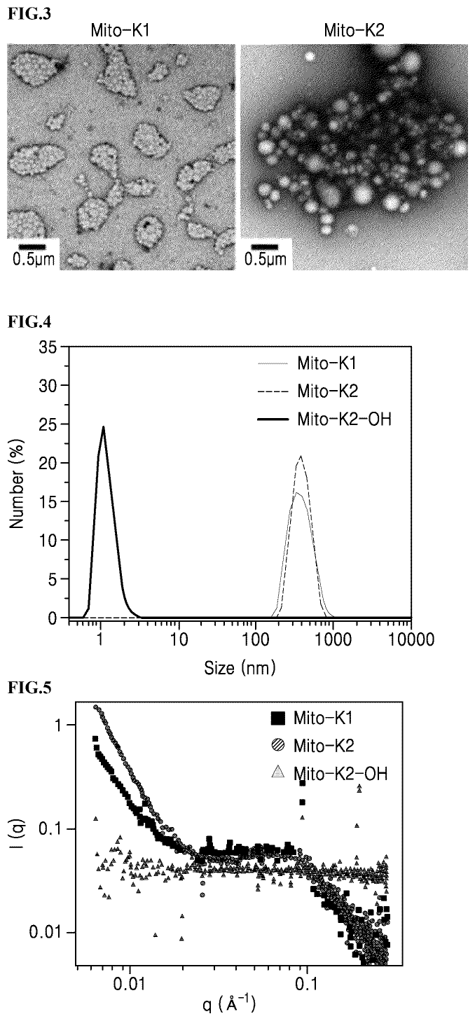

A 5 mM aqueous solution of Mito-K1 and Mito-K2 was stirred at 37° C. for 24 hours in mitochondrial-mimicking solution under oxidizing conditions (pH 8.0 PBS containing 10 mM GSH and 1 mM H₂O₂). Whether oligomerization occurred was determined by matrix-assisted laser desorption/ionization time-of-flight (MALDI-TOF) mass spectrometry, and the results are shown in FIG. 1. In the same manner, an analysis was performed under conditions without hydrogen peroxide (H₂O₂), and the results are shown in FIG. 2.

As shown in FIGS. 1 and 2, oligomers ranging from dimers to decamers were observed in the Mito-K2 solution. Although dimers, trimers, and tetramers were predominant in the Mito-K1 solution, showing a similar trend, only monomer peaks were observed in the Mito-K2-OH solution.

These results confirmed that peptide oligomerization occurred even under reducing conditions, induced by disulfide bond formation via an oxidant.

### 1.2. Confirmation of Self-Assembled Polymer Formation

Using the same method as in Experimental Example 1.1, a 5 mM aqueous solution of Mito-K1 and Mito-K2 was stirred at 37° C. for 24 hours, and self-assembled polymer formation was confirmed by transmission electron microscopy (TEM) and scanning electron microscopy; and the results are shown in FIG. 3. To confirm the nanostructure formation of the oligomers, dynamic light scattering (DLS) analysis and small-angle X-ray scattering (SAXS) analysis were performed, and each result is shown in FIGS. 4 and 5, respectively.

As shown in FIG. 3, both Mito-K1 and Mito-K2 exhibited aggregation into spherical structures, whereas Mito-K2-OH did not.

As shown in FIG. 4, both Mito-K1 and Mito-K2 solutions exhibited nanostructures around 500 nm, whereas only insignificant aggregates of approximately 1 nm were observed with Mito-K2-OH.

As shown in FIG. 5, both Mito-K1 and Mito-K2 formed disordered block aggregates.

These results indicate that, under oxidizing conditions, Mito-K1 and Mito-K2 form self-assembled polymers, and that the oligomers aggregate into spherical structures to form nanostructures.

### 1.3. Confirmation of the Secondary Structure of the Self-Assembled Polymers

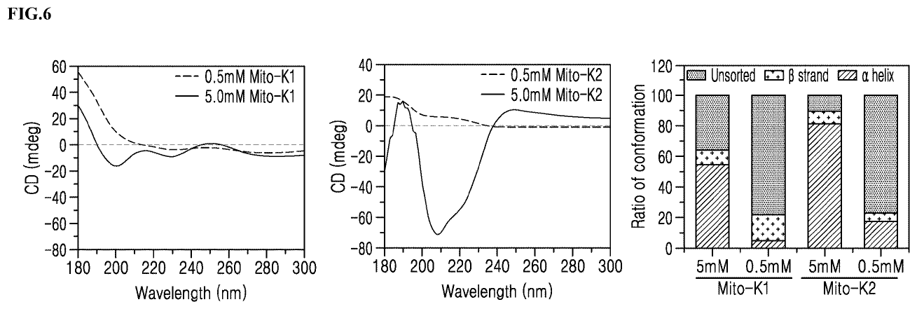

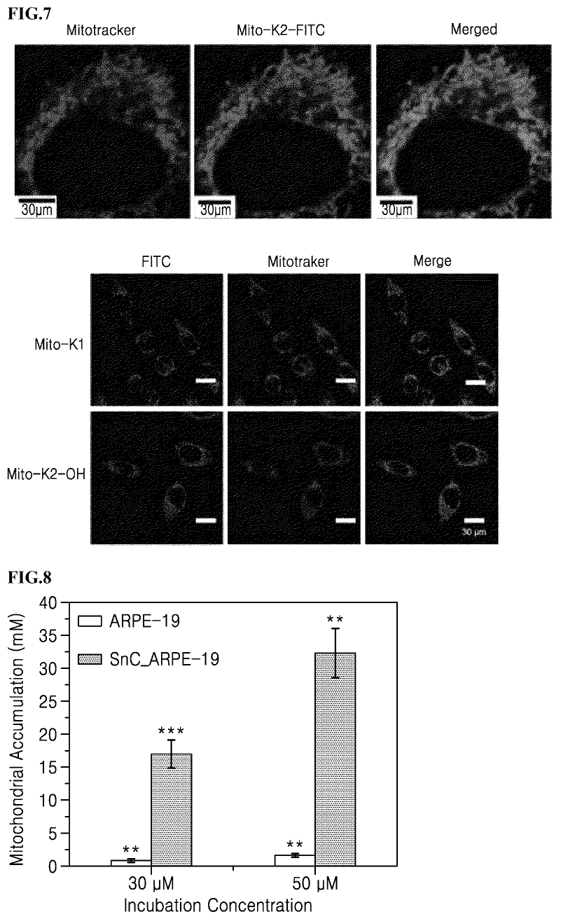

To confirm the secondary structures in Mito-K1 and Mito-K2 solutions, 0.5 mM and 5 mM aqueous solutions of Mito-K1 and Mito-K2 were analyzed using circular dichroism (CD), and the results are shown in FIG. 6.

As shown in FIG. 6, the α-helix structure was clearly observed only in the 5 mM solutions, and it was far more predominant in the cationic Mito-K2 compared to Mito-K1. Specifically, the degree of α-helix structure formation was measured to be 0.8 and 0.5 for Mito-K2 and Mito-K1, respectively.

These results indicate that the formation of self-assembled polymers, induced by oligomerization, leads to the formation of α-helical secondary structures.

### Experimental Example 2. Confirmation of Oligomerization and Self-Assembled Polymer Formation in Mitochondria

### 2.1. Confirmation of Senescence Induction by Doxorubicin Treatment

ARPE-19 cells, a human retinal pigment epithelium (RPE) cell line, were induced to senescence using doxorubicin (Dox), an agent used to induce cellular senescence by generating superoxide anions and causing DNA damage. To confirm the induction of senescence, senescence-induced ARPE-19 cells were stained for Senescence-associated beta-galactosidase (SA-β-gal), confirming that over 95% of the cells were senescent. It was also confirmed that senescence-induced ARPE-19 cells overexpressed integrin avB3 on their cell membrane, exhibiting a phenotype distinct from that of normal cells.

### 2.2. Confirmation of Mitochondrial Targeting of Mito-K1 and Mito-K2

To measure the concentration of Mito-K2 inside mitochondria, the following experiment was performed.

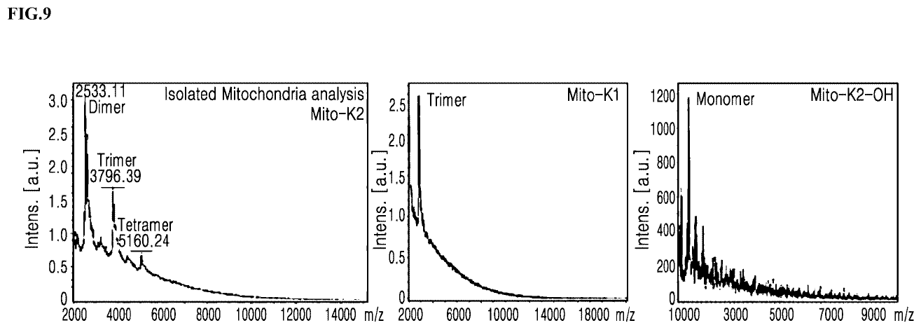

Dox-induced senescent cells were cultured for 24 hours in a 6-well plate to allow cell attachment, and then treated with Mito-K2-FITC for 12 hours. The cells were then harvested using trypsin-EDTA and washed three times with cold PBS. The cell count was determined using a LUNAR cell counter, and mitochondria were isolated using a mitochondrial isolation kit (Thermofisher). The isolated mitochondria were lysed with RIPA cell lysis buffer, and the fluorescence of the solution was measured using a fluorometer. The concentration of Mito-K2-FITC inside the mitochondria was measured based on the maximum emission at 525 nm and a calibration curve. Mito-K1-FITC and Mito-K2-OH-FITC were treated and their concentrations measured in the same manner, and the results are shown in FIG. 7.

In the same manner, ARPE-19 cells and Dox-induced senescent ARPE-19 cells were treated with 30 M and 50 M of Mito-K2-FITC, respectively, and then imaged by confocal microscopy. The results are shown in FIG. 8.

As shown in FIG. 7, the green fluorescence of Mito-K2-FITC, Mito-K1-FITC, and Mito-K2-OH-FITC co-localized with a marker that selectively stains mitochondria.

As shown in FIG. 8, incubation with 30 UM and 50 UM of Mito-K2-FITC resulted in accumulation of 16.95 mM and 32.42 mM, respectively, of Mito-K2-FITC inside the mitochondria of the senescent cells. In normal RPE cells, even after incubation with 50 HM Mito-K2-FITC, the intracellular mitochondrial concentration remained below 2 mM.

Considering these findings and the fact that the critical monomer concentrations of Mito-K1 and Mito-K2 required for nanostructure formation in a reducing environment are between 2.24 mM and 2.56 mM, it was confirmed that supplying the peptide at a concentration below 50 UM selectively induces the formation of artificial nanostructures, comprising self-assembled polymers, within the mitochondria of senescent RPE cells.

### 2.3. Confirmation of Oligomer Formation Within Mitochondria

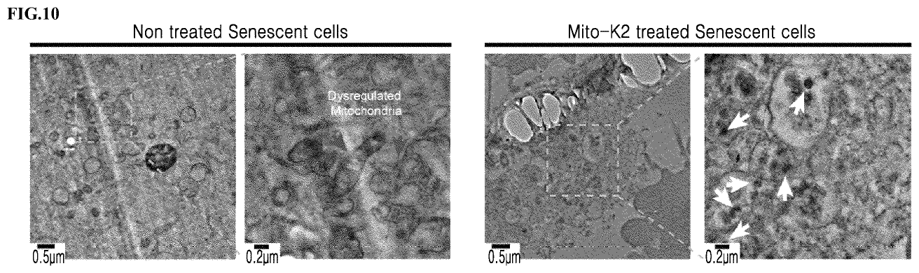

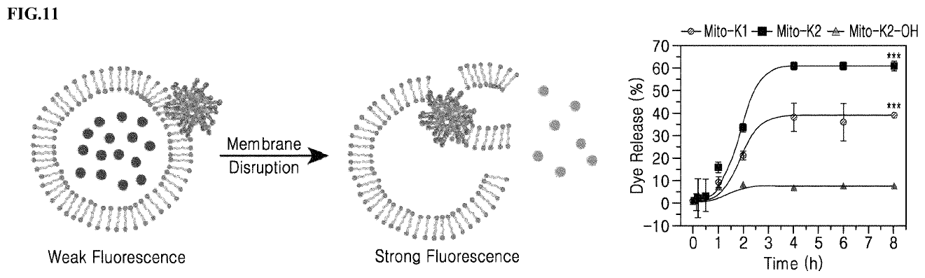

The Dox-induced senescent RPE cells were treated with 50 UM Mito-K2 and incubated for 12 hours. Mitochondria were isolated as in Experimental Example 2.2, and the resulting mitochondrial solution was analyzed by matrix-assisted laser desorption/ionization time-of-flight (MALDI-TOF) mass spectrometry. Mito-K1 and Mito-K2-OH were analyzed in the same manner, and the results are shown in FIG. 10.

Normal RPE cells and Dox-induced senescent RPE cells were treated with Mito-K2 and incubated for 12 hours, after which mitochondria were observed via transmission electron microscopy (TEM). Those images are presented in FIG. 11.

As shown in FIG. 10, oligomers were predominantly detected inside the mitochondria of cells treated with Mito-K2, whereas trimers were predominantly observed in cells treated with Mito-K1; however, only monomer peaks were detected with Mito-K2-OH.

As shown in FIG. 11, damaged mitochondria with disrupted cristae and a swollen morphology were observed in Dox-induced senescent RPE cells.

These results indicate that oligomerization occurs selectively only within the mitochondria of Dox-induced senescent RPE cells.

### Experimental Example 3. Disruption of Senescent Cell Mitochondria by Intracellular Oligomerization

### 3.1. Confirmation of the Interaction Between Intracellular Nanostructures and Mitochondrial Membranes

To confirm the interaction between mitochondrial membranes and nanostructures formed by oligomerization of self-assembled polymers, the following experiment was performed.

Liposomes mimicking the mitochondrial membrane were loaded with the fluorescent dye calcein. Subsequently, the liposomes were treated with 10 mM of Mito-K1 and Mito-K2, respectively. Whether the oligomers formed by the self-assembly process could disrupt the liposomes was assessed by measuring the release of the fluorescent dye, and a schematic diagram and the results of the experiment are shown in FIG. 11.

As shown in FIG. 11, up to 60% leakage was observed upon treatment with Mito-K2, and up to 35% leakage was measured upon treatment with Mito-K1. In contrast, no significant release of the fluorescent dye was observed in liposomes treated with Mito-K2-OH.

These results suggest that the nanostructures formed inside the mitochondria of senescent cells interact with the mitochondrial membrane, causing mitochondrial membrane dysfunction.

### 3.2. Disruption of Mitochondrial Membrane

To assess mitochondrial membrane depolarization, the following experiment was performed.

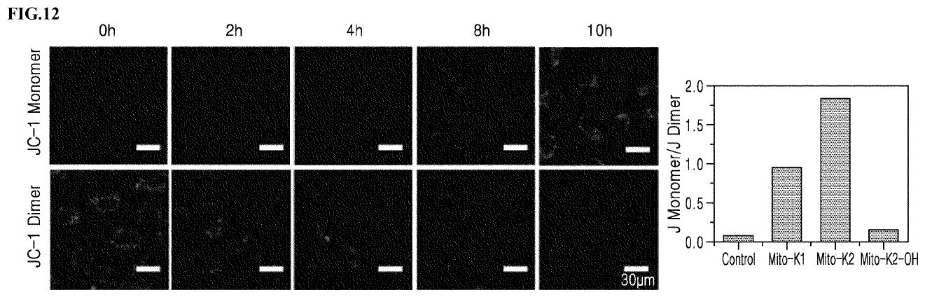

Normal RPE cells and Dox-induced senescent RPE cells were cultured in cell culture medium in an 8-well plate (Thermofish) until reaching 80% confluence. After treatment with 80 μM Mito-K2 for 10 hours, the cells were washed three times with cold PBS. Depolarized mitochondrial membranes were detected by treatment with 2 μM JC-1 for 30 minutes, and JC-1-associated cells were monitored using an LSM 7000. The same procedure was performed with Mito-K1 and Mito-K2-OH. In intact mitochondria, the J-probe aggregates and emits red fluorescence; if membrane depolarization occurs, green fluorescence is emitted. The results are shown in FIG. 12.

As shown in FIG. 12, red fluorescence was observed at 0 h and 2 h, but green fluorescence was observed at 10 h. In addition, Mito-K2 caused more severe damage compared to Mito-K1, whereas Mito-K2-OH did not display any indicators of mitochondrial damage.

These results indicate that Mito-K2 disrupts mitochondrial membranes.

### 3.3. Mitochondrial Damage-ROS Generation, Morphological Changes, and ATP Level Measurement

To assess mitochondrial membrane damage in senescent cells, the following experiment was performed.

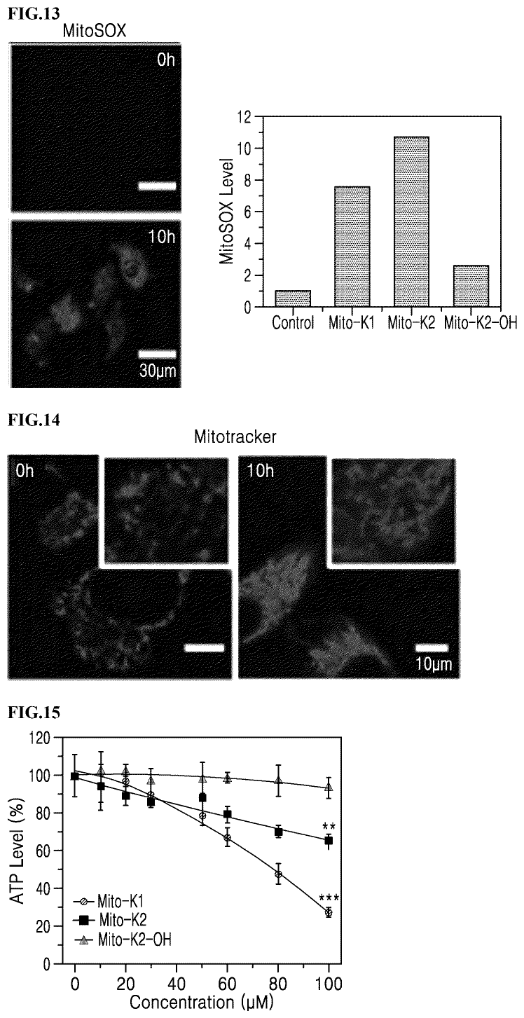

Mitochondrial membrane depolarization in senescent cells leads to oxidative stress on the mitochondria, which induces ROS formation. After mitochondrial membrane depolarization occurred in Dox-induced senescent RPE cells treated with Mito-K2, the cells were treated with MitoSOX—which emits red fluorescence upon elevated ROS levels—to confirm ROS generation. Subsequently, the cells were analyzed by immunofluorescence, and the results are shown in FIG. 13.

To observe mitochondrial morphology following Mito-K2 treatment, immunostaining was performed using MitoTracker Deep Red, a dye that selectively stains mitochondria. Senescent cells stained with MitoTracker Deep Red were analyzed by confocal laser scanning microscopy (CLSM), and the results are shown in FIG. 14.

Mitochondrial dysfunction was assessed by measuring ATP levels. ATP levels were monitored in Dox-induced senescent RPE cells following treatment with Mito-K1, Mito-K2, and Mito-K2-OH, and the results are shown in FIG. 15.

As shown in FIG. 13, self-assembled polymer formation within the mitochondria induced oxidative stress.

As shown in FIG. 14, mitochondria in senescent cells treated with Mito-K2 were fragmented and swollen in appearance.

As shown in FIG. 15, ATP levels decreased by approximately 80% in senescent cells treated with Mito-K2 and by approximately 30% in cells treated with Mito-K1. In contrast, no significant change in ATP levels was observed following treatment with Mito-K2-OH.

These results indicate that the morphology and function of mitochondria were damaged in senescent cells treated with Mito-K1 and Mito-K2.

### Experimental Example 4 Elimination of Senescent Retinal Pigment Epithelial Cells by Mito-K2

### 4.1. Confirmation of Cytotoxicity of Mito-K2 and Mito-K1

To assess the toxicity of Mito-K2 and Mito-K1 on both normal RPE cells and Dox-induced senescent RPE cells, the following experiment was performed.

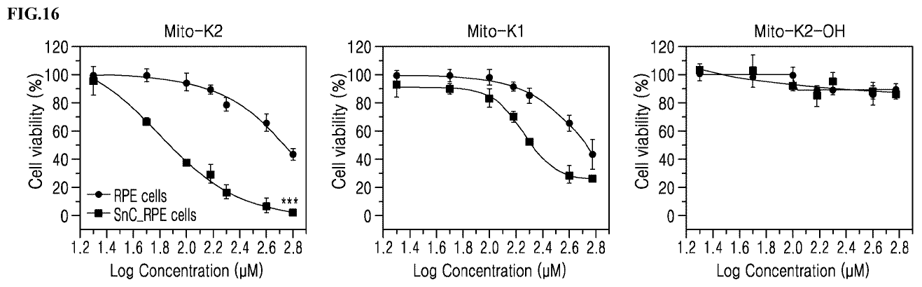

Normal RPE cells and Dox-induced senescent RPE cells were incubated in a 96-well plate (Thermofisher) to maintain 80% confluence in cell culture medium. After incubation, different concentrations of Mito-K2 were applied to each cell type at 24 hours and 48 hours, and cell viability was assessed using an MTT assay. The MTT luminescence was analyzed using a microplate reader, and the results are shown in FIG. 16.

As shown in FIG. 16, Mito-K2 exhibited selectively high toxicity only towards senescent cells, whereas Mito-K1 showed lower toxicity towards senescent cells compared to Mito-K2, and Mito-K2-OH showed no toxicity towards either normal or senescent cells.

### 4.2. Confirmation of the Apoptosis-Inducing Effect of Mito-K2

To determine whether Mito-K2 treatment selectively induces apoptosis in senescent cells, the following experiment was performed.

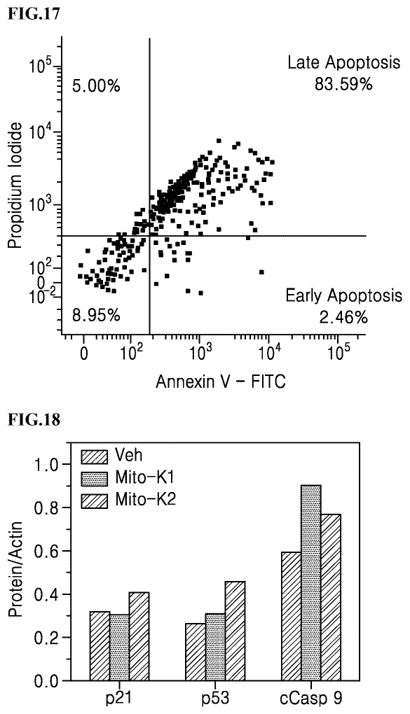

Normal RPE cells and senescence-induced RPE cells were treated with Mito-K2, and after 24 hours, were treated with Annexin V-FITC. At this point, cells undergoing apoptosis selectively exhibit green fluorescence. The detected green fluorescence was analyzed by fluorescence-activated cell sorting (FACS), and the results are shown in FIG. 17.

Additionally, the following experiment was performed to confirm whether p53, p21, and cCasp 9 are overexpressed in Dox-induced senescent RPE cells.

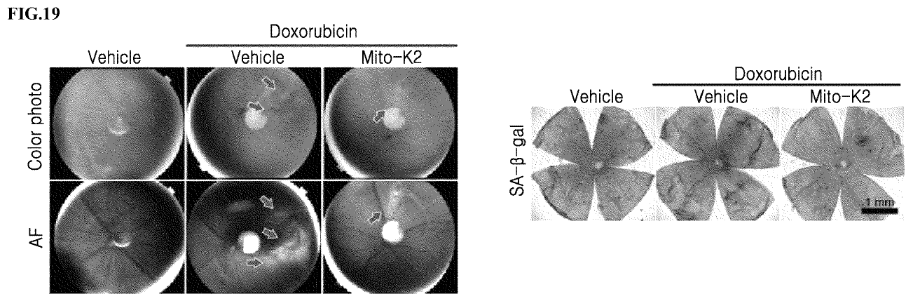

Normal RPE cells and senescence-induced RPE cells were lysed in RIPA buffer (Thermo Fisher Scientific, 89901) supplemented with a protease inhibitor cocktail (Roche, 11697498001, Basel, Switzerland). The protein concentration of the cell lysates was quantified using a BCA assay (Pierce, 23227, Waltham, USA) or a Bradford protein assay (Bio-Rad). Thereafter, an equal amount of protein was separated by SDS-PAGE on a polyacrylamide gel and transferred to a polyvinylidene difluoride (PVDF) membrane. The PVDF membrane was then blocked for 2 hours in 5% skim milk in TBS-T buffer and incubated overnight at 4° C. with primary antibodies, including anti-cleaved caspase-9 (1:1000, Abcam, ab2324), anti-p53 (1:250, Abcam, ab90363), and anti-p21 (1:1000, Abcam, ab109520). This was followed by a 2-hour incubation with a horseradish peroxidase-conjugated secondary antibody. Subsequently, immunoreactive proteins were visualized using a chemiluminescent substrate (Amersham, RDN2232, Little Chalfont, UK) and quantified by densitometry with ImageJ software (NIH, USA). All experiments were repeated at least three times, and the results are shown in FIG. 18.

As shown in FIG. 17, senescent cells were confirmed to enter the apoptotic stage within 12 hours after treatment with Mito-K2.

As shown in FIG. 18, Mito-K2 and Mito-K1 increased the levels of p53 and pro-caspase-9. This indicates that treatment with Mito-K2 or Mito-K1 induces apoptosis in senescent cells.

### 4.3. Confirmation of the Inhibitory Effect of Mito-K2 on Senescent Cell Induction In Vivo

Using doxorubicin (Dox) as a senescence-inducing agent, mouse retinal tissue was induced to senescence as follows.

Specifically, doxorubicin was injected into the subretinal space of a mouse eye to induce senescence in RPE cells. On day 0, 100 ng/μL of doxorubicin was injected; Mito-K2 was intravitreally injected at a dose of 253.2 ng/μL on day 0 and day 3. All evaluations of the in vivo Dox-induced senescent RPE cells were performed on day 7.

Under an optical microscope (Olympus SZ51, Tokyo, Japan), a small hole was made at the limbus using a 30-gauge sterile needle (BD Science, San Jose, USA). A blunt 35-gauge Hamilton microsyringe (Hamilton Company, NV, USA) was then slowly inserted through the hole. Subsequently, 100 ng/μL of Dox was injected into the subretinal space of C57BL/6J mice.

Then, 1 μL of either control reagent (1× phosphate-buffered saline) or 253.2 ng/μL of Mito-K2 was injected using a blunt 35-gauge Hamilton microsyringe. At the conclusion of the experiment, the mice were anesthetized, and their eyes were immediately enucleated. First, the anterior segment was removed with surgical micro-scissors, and the retina was carefully dissected out under an optical microscope (Olympus SZ51, Tokyo, Japan). Consequently, the RPE/choroid/sclera complex tissue was washed with cold 1× PBS. After dilating the eyes, color fundus photography and fundus autofluorescence images of the mouse eyes were captured using a TRC-50 DX camera (Topcon, Tokyo, Japan) connected to a digital imaging system (IMAGEnet 6 Version, Topcon), and the images are shown in FIG. 19.

Senescence was assessed using an SA-β-galactosidase staining kit (#9860, Cell Signaling Technology, Danvers, MA, USA). Briefly, the RPE/choroid/sclera complex tissue was fixed in 1× fixative for 30 minutes, washed with PBS, and then incubated overnight at 37° C. in SA-β-gal staining solution. To quantify the stained SA-β-galactosidase, the RPE/choroid/sclera complex tissue was bleached by immersion in 10% H₂O₂ and incubated in a heat block at 55° C. for 45 minutes, rinsed with PBS, and then flat-mounted under an optical microscope (Olympus SZ51) using Aqua Poly/Mount (#18,606-20, Polysciences, Inc., Warrington, PA, USA). The stained RPE/choroid flat mounts were captured using an inverted microscope (Carl Zeiss Axio Scope A1, Gottingen, Germany), and the images are shown in FIG. 19.

As shown in FIG. 19, cells in the mouse retinal tissue were induced to senescence, and Mito-K2 was confirmed to suppress the induction of senescence.

### 4.4. Confirmation of the Inhibitory Effect of Mito-K2 on ROS Overexpression in Senescent Cells In Vivo

To assess whether Mito-K2 suppresses ROS overexpression in senescent cells induced in Experimental Example 4.3, the following experiment was performed.

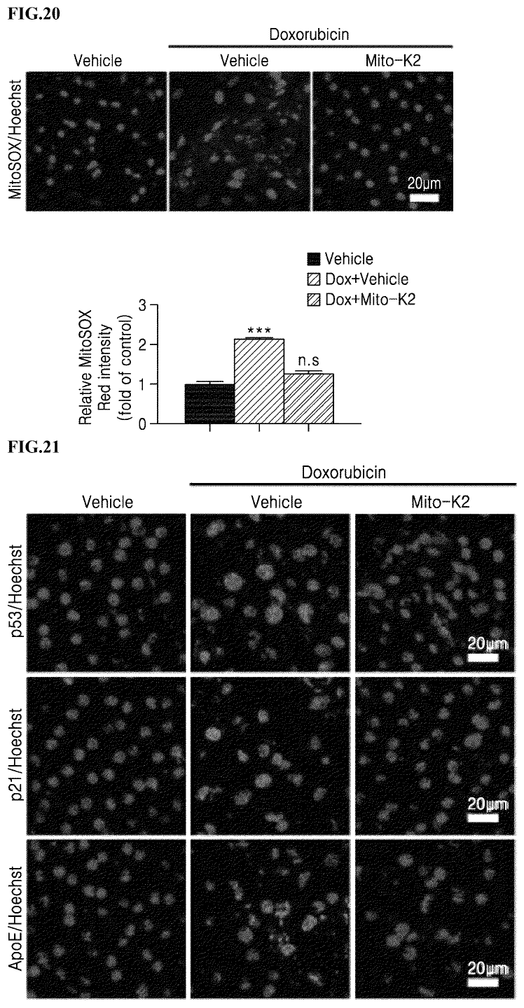

In RPE/choroid flat mounts from mice, MitoSOX was incubated for 10 minutes in a humidified incubator at 37° C. and 5% CO₂, followed by staining of the tissue with Hoechst 33342 (1:1,000, Thermofish) in PBS for 15 minutes. The tissue was then mounted using a mounting solution, and mitochondrial ROS in live cells were observed using an inverted microscope (LSM 900, Carl Zeiss). The results are shown in FIG. 20.

As shown in FIG. 20, while ROS were overexpressed in senescent cells, Mito-K2 suppressed ROS expression to a level similar to that of the control group.

4.5. Confirmation of the Inhibitory Effect of Mito-K2 on p53, p21, and ApoE Overexpression in Senescent Cells In Vivo

To assess whether Mito-K2 inhibits the overexpression of p53, p21, and ApoE in senescent cells induced in Experimental Example 4.3, the following experiment was performed.

Mouse RPE/choroid flat mounts were permeabilized with 0.1% Triton X-100 in PBS for 15 minutes. Subsequently, the tissue was blocked in 1% BSA in PBS for 1 hour, and the fixed tissue or cells were incubated overnight at 4° C. with primary antibodies against p53 (1:250, Santa Cruz Biotechnology, TX, USA), p21 (1:500, Santa Cruz Biotechnology), and APOE (1:500, Santa Cruz Biotechnology). The stained tissue or cells were then washed with PBS and incubated for 2 hours at room temperature with Alexa Fluor-conjugated secondary antibodies. The secondary antibodies used were Alexa Fluor 555-conjugated goat anti-mouse IgG (Thermo Fisher Scientific, A-11029, Waltham, USA) or Alexa Fluor 555-conjugated goat anti-rabbit IgG (Thermo Fisher Scientific, A-21424). All secondary antibodies were used at a 1:250 dilution. After incubation with the secondary antibodies, the tissue or cells were stained with the nuclear dye Hoechst 33342 (1:1000, Thermo Fisher Scientific, H3570) in PBS for 15 minutes at room temperature. The tissue was then mounted using a mounting medium (Polyscience). The stained cells were observed under an inverted microscope (Carl Zeiss, LSM 900, Oberkochen, Germany), and the results are shown in FIG. 21.

As shown in FIG. 21, p53, p21, and APOE were overexpressed in senescent cells, but Mito-K2 suppressed the expression of p53, p21, and APOE to levels comparable to those of the control group.

### 4.6. Confirmation of the Inhibition of Overexpression of Senescence Markers and SASP Factors in Senescent Cells In Vivo

To assess whether Mito-K2 inhibits the elevated levels of senescence-associated secretory phenotype (SASP) factors in senescent cells induced in Experimental Example 4.3, the following experiment was performed.

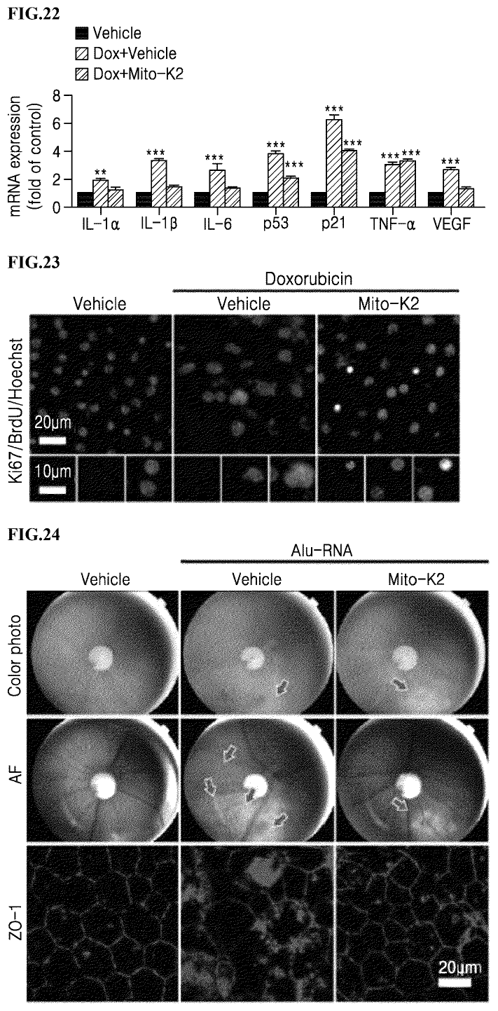

Total RNA was isolated from mouse RPE cells using TRIzol reagent (Invitrogen, 15596026). After RNA isolation, mRNA was reverse transcribed into cDNA using a reverse transcriptase (Thermo Fisher Scientific, 4368813). The mRNA was quantified using specific primers with 40 cycles of the CFX Connect Real-Time System (BIO-RAD, CA, USA). The PCR products were identified by melting-curve analysis. Relative mRNA expression was calculated according to the 2^{-ΔΔCt} method. GAPDH was used as an internal reference gene. The average Ct value was normalized to GAPDH. Real-time PCR was performed at least three times for each group, and the results are shown in FIG. 22.

As shown in FIG. 22, while SASP factors were overexpressed in senescent cells, Mito-K2 suppressed the expression of these SASP factors to levels comparable to those of the control group.

### 4.7. Confirmation of the Elimination of Senescent RPE Cells and Proliferation of Normal RPE Cells In Vivo

To assess the effect of Mito-K2 on increasing the proliferative activity of senescent cells induced in Experimental Example 4.3, the following experiment was performed.

A BrdU solution (200 μL, 5 mg/mL; #B5002, Sigma-Aldrich, MO, USA) was injected intraperitoneally using a 1 ml syringe attached to a 30-gauge sterile needle, after which the eyes were enucleated. The RPE/choroid flat mounts were incubated in 2 N HCl at 37° C. for 30 minutes, then washed twice for 5 minutes each with 0.1 M borate buffer (pH 8.5). The fixed samples were blocked in 1% BSA in PBS for 1 hour, and then incubated overnight at 4° C. with primary antibodies against BrdU (1:1000, Invitrogen, MA3-071, CA, USA) and Ki67 (1:1000, Cambridge, UK). The tissue was then washed with PBS and incubated for 2 hours at room temperature with Alexa Fluor-conjugated secondary antibodies, specifically, Alexa Fluor 488-conjugated goat anti-rabbit IgG (Thermo Fisher Scientific, A-11034) or Alexa Fluor 555-conjugated goat anti-mouse IgG (Thermo Fisher Scientific, A-11029). The results are shown in FIG. 23.

As shown in FIG. 23, expression of Ki67 and BrdU was observed only in senescent cells treated with Mito-K2. This indicates that Dox-induced senescent RPE cells were eliminated by Mito-K2, and the number of normal RPE cells increased.

### Experimental Example 5 Elimination of Senescent Retinal Pigment Epithelial Cells Using Mito-K2 in a Mouse Model of Macular Degeneration

### 5.1. Confirmation of the Inhibitory Effect of Mito-K2 on Senescent Cell Induction In Vivo

A mouse model of macular degeneration (geographic atrophy, GA model) was induced as follows, using Alu RNA as a senescent cell-inducing agent.

Specifically, Alu RNA was injected into the subretinal space of a mouse eye to induce a GA model of macular degeneration. On day 0, 2.4 μg/μL of Alu RNA was injected; Mito-K2 was intravitreally injected at a dose of 253.2 ng/μL on day 0 and day 3. All evaluations regarding the induced macular degeneration mouse model were performed on day 7.

Color fundus photography and fundus autofluorescence images of the eyes of the macular degeneration mouse model were captured in the same manner as in Experimental Example 4.3. The results are shown in FIG. 24.

The RPE/choroid flat mounts from the mice were permeabilized with 0.1% Triton X-100 in PBS for 15 minutes. Subsequently, the tissue was blocked in 1% BSA in PBS for 1 hour, and the fixed tissue or cells were incubated overnight at 4° C. with a primary antibody against ZO-1 (1:1,000, Invitrogen). The stained tissue or cells were then washed with PBS and incubated for 2 hours at room temperature with Alexa Fluor-conjugated secondary antibodies. The secondary antibodies used were Alexa Fluor 555-conjugated goat anti-mouse IgG (Thermo Fisher Scientific, A-11029, Waltham, USA) or Alexa Fluor 555-conjugated goat anti-rabbit IgG (Thermo Fisher Scientific, A-21424). The secondary antibodies were used at a 1:250 dilution. The tissue was then mounted using a mounting medium (Polyscience). The stained cells were observed under an inverted microscope (Carl Zeiss, LSM 900, Oberkochen, Germany), and the results are shown in FIG. 24.

As shown in FIG. 24, cells in the mouse retinal tissue were induced to senescence, and Mito-K2 was confirmed to inhibit the induction of senescence.

### 5.2. Confirmation of Inhibitory Effect of Mito-K2 on ROS Overexpression in Senescent Cells In Vivo

To assess whether Mito-K2 inhibits ROS overexpression in retinal cells of mice with Alu RNA-induced macular degeneration as described in Experimental Example 5.1, the following experiment was performed.

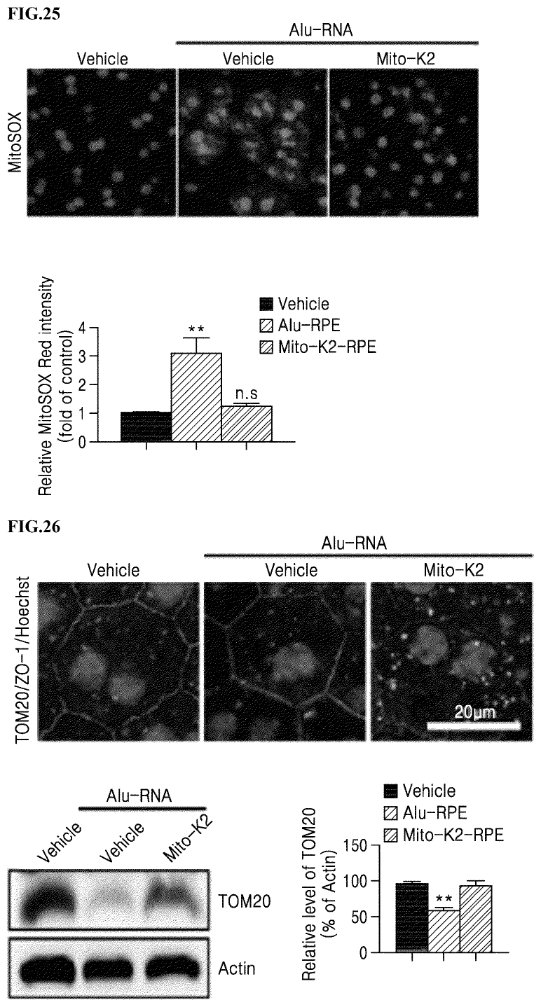

MitoSOX expression in the retinal tissue cells of mice with induced macular degeneration was measured as described in Experimental Example 4.5, and the results are shown in FIG. 25.

As shown in FIG. 25, while the retinal tissue cells of the mice overexpressed ROS, Mito-K2 suppressed ROS expression to a level similar to that of the control group.

### 5.3. Confirmation of the Inhibitory Effect of Mito-K2 on TOM20 Overexpression in Alu-Induced Senescent Cells In Vivo

To assess whether the mouse cells induced with macular degeneration by Alu RNA in Experimental Example 5.1 overexpress TOM20 despite the influence of Mito-K2, the following experiment was performed.

Mouse RPE/choroid flat mounts were permeabilized with 0.1% Triton X-100 in PBS for 15 minutes. Subsequently, the tissue was blocked in 1% BSA in PBS for 1 hour, followed by overnight incubation at 4° C. with primary antibodies against TOM20 (1:500, Cell Signaling) and ZO-1 (1:1,000, Invitrogen). The stained tissue or cells were then washed with PBS and incubated for 2 hours at room temperature with Alexa Fluor-conjugated secondary antibodies. The secondary antibodies used were Alexa Fluor 555-conjugated goat anti-mouse IgG (Thermo Fisher Scientific, A-11029, Waltham, USA) or Alexa Fluor 555-conjugated goat anti-rabbit IgG (Thermo Fisher Scientific, A-21424). All secondary antibodies were used at a 1:250 dilution. After incubation with the secondary antibodies, the tissue or cells were stained with the nuclear dye Hoechst 33342 (1:1,000, Thermo Fisher Scientific, H3570) in PBS for 15 minutes at room temperature. Subsequently, the tissue was mounted using a mounting medium (Ployscience). The stained cells were then observed under an inverted microscope (Carl Zeiss, LSM 900, Oberkochen, Germany). The results are shown in FIG. 26.

As shown in FIG. 26, while the retinal tissue cells of the mice overexpressed TOM20, Mito-K2 suppresses TOM20 expression to a level similar to that of the control group.

### 5.4. Confirmation of Mitochondrial Function Ex Vivo

Mitochondrial function was assessed by measuring the oxygen consumption rate (OCR) of retina and RPE cells, ex vivo, as follows.

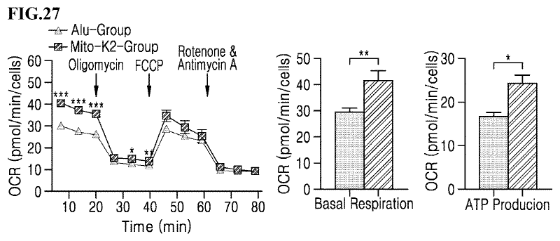

The OCR of the retina/RPE cells was measured using an XFe96 analyzer (Agilent Technologies). One day prior to measurement, the sensor cartridge of the Seahorse XF Calibrant (Agilent Technologies) was hydrated overnight in a non-CO₂ 37° C. incubator, using Seahorse XF Calibrant (Agilent Technologies). Mouse retinal tissue and RPE cells were isolated from enucleated eyeballs and dissociated using a papain dissociation system. The dissociated cells were suspended in XF assay medium and dispensed into an XF96 cell culture microplate (Agilent Technologies) coated with Matrigel (#356230, growth factor-reduced Matrigel, Corning). After measuring basal respiration for 18 minutes, mitochondrial complex inhibitors (oligomycin, FCCP, rotenone, and antimycin A) were sequentially added to the XF assay medium. Oligomycin (1.5 μM) was injected into each well, followed by FCCP (1 μM) at 36 minutes, and rotenone (0.5 μM) plus antimycin A (0.5 μM) at 54 minutes. The oxygen consumption rate was measured in pMole/min, and changes in ATP levels were measured after oligomycin treatment. Each data point was derived from at least three wells, and the results are shown in FIG. 27.

As shown in FIG. 27, it was confirmed that basal respiration and ATP production were significantly increased in cells from the retinal tissue of mice administered Mito-K2. This indicates that Mito-K2 enhances mitochondrial function.

### Experimental Example 6. Single-Cell RNA Sequencing Revealing the Effects of Mito-K2 in a Macular Degeneration Mouse Model

To further assess the therapeutic effects of Mito-K1 and Mito-K2 against senescence, the following experiment was performed.

### 6.1. Single-Cell RNA Sequencing

Single-cell RNA sequencing was performed on retinal/RPE/choroid tissue from three groups: a control group, an Alu group (subretinal injection of Alu and intravitreal injection of vehicle), and a Mito-K2 group (subretinal injection of Alu and intravitreal injection of Mito-K2).

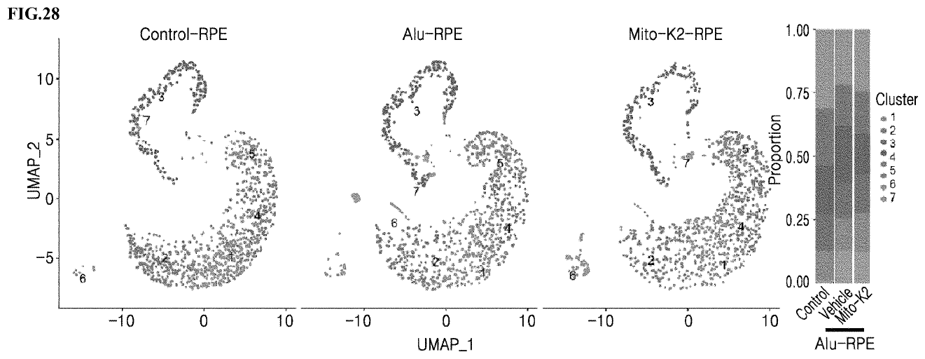

The cell suspension was mixed with a reverse transcription master mix and loaded onto a single-cell K Chip, along with single-cell 5′ Gel Beads and Partitioning Oil. The RNA transcripts from each single cell were uniquely barcoded and reverse transcribed. The resulting cDNA was then pooled, concentrated, and amplified by PCR to generate a 5′ gene-expression library. The purified library was quantified by qPCR and qualitatively assessed using an Agilent Technologies 4200 TapeStation (Agilent Technologies). The library was then sequenced using a HiSeq platform (Illumina), generating 150 bp paired-end reads. Analysis of 41,979 cells using UMAP yielded a clustering result with eight clusters showing distinct expression profiles. Subsequently, the major cell type of each cluster was identified by comparing the expression levels of known, cell-type-specific markers to those of the representative genes expressed in each cluster. A total of 7,011 RPE cells were identified based on the expression of RPE-specific markers, including RPE65 (control-RPE: 3,424 cells, Alu-RPE: 3,587 cells). The 7,011 RPE cells from the control and Alu groups were further subdivided into seven clusters, and the clustering results are shown in FIG. 28.

As shown in FIG. 28, the distribution of cell numbers within each cluster differed among the control, Alu, and Mito-K2 groups.

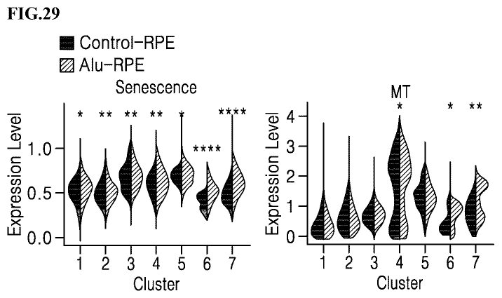

To determine the function associated with each cluster, gene ontology (GO) analysis was performed using the top representative markers of each cluster. To identify Alu-induced senescent RPE cells, a “senescence panel” of 12 genes—A2m, Ccng1, Cdkn1a, Ckmt1, Nrtk2, Clu, Gas6, Malat1, Serping1, Tmem176b, Vim, and Timp1—was selected based on recent literature reviews and studies, and differences in gene expression between the control and Alu groups were assessed. Similarly, a “mitochondrial panel” of nine genes—mt-Atp6, mt-Atp8, mt-Co1, mt-Co2, mt-Co3, mt-Nd1, mt-Nd2, mt-Nd3, and mt-Nd41—was selected to assess gene expression differences between the control and Alu groups, and the results are shown in FIG. 29.

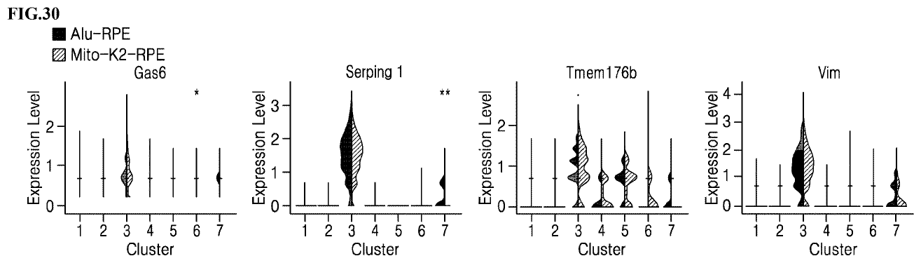

Using the same approach, changes in expression of four genes (Gas6, Serping1, Tmem176a, and Vim) belonging to the senescence panel were analyzed on a cluster-by-cluster basis in the Alu group and the Mito-K2 group, and the results are shown in FIG. 30.

As shown in FIG. 29, both the senescence panel and the mitochondrial panel showed a marked increase in cell numbers in clusters 6 and 7. This suggests a strong likelihood that both Alu-induced senescent cells and non-senescent cells are mixed within clusters 6 and 7.

As shown in FIG. 30, in the Mito-K2 group, the expression of genes belonging to the senescence panel was downregulated in the cell population of cluster 7.

These results indicate that senescent RPE cells are selectively eliminated in cluster 7.

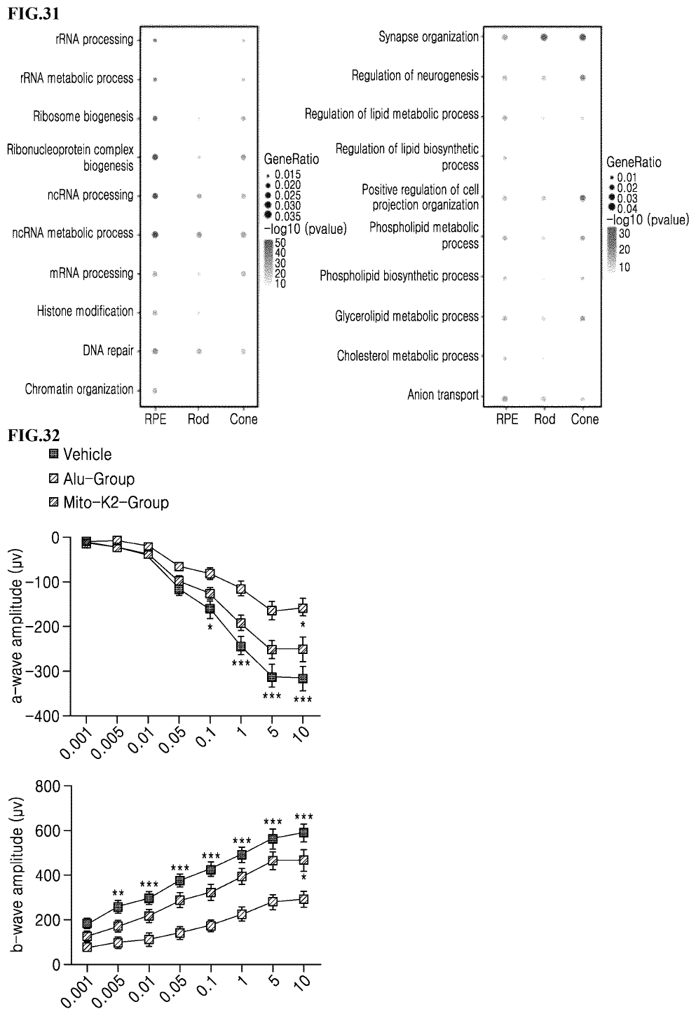

To examine the effects of Mito-K2 on changes in gene function in the Alu RNA-induced retinal degeneration mouse model, gene ontology (GO) analysis was performed on RPE cells, rod cells, and cone cells, and the results are shown in FIG. 31.

As shown in FIG. 31, genes that had been downregulated in the Alu group were upregulated by Mito-K2 (right), whereas genes that had been upregulated in the Alu group were downregulated by Mito-K2 (left).

### 6.2. In Vivo Electroretinogram (ERG) Measurement

Mitochondrial function was assessed by measuring the oxygen consumption rate (OCR) of retina and RPE cells, ex vivo, as follows.

To assess retinal function, an electroretinogram (ERG) was recorded using a Celeris rodent ERG system (Diagnosys, LLC, MA, USA). Prior to the experiment, mice were dark-adapted for at least 16 hours, then anesthetized via intraperitoneal injection of a mixture of Zoletil (Virbac, France) and xylazine (Rompun, Bayer HealthCare, Leverkusen, Germany) diluted 4:1 in physiological saline. The pupils were dilated with topical Tropherine eye drops (Hanmi, Korea) and 2% hypromellose (Samil, Korea), and electrodes were subsequently inserted. The experiment was performed under dim red light to maintain the mouse's body temperature. For ERG measurement, an electrode was placed on the cornea in the center of the pupil, and the reference electrode was placed on the contralateral eye. Dark-adapted ERG responses were measured using single-flash stimuli ranging from 0.001 cd·s/m² to 10 cd·s/m², and each flash intensity was recorded as at least three responses. Amplitudes of a- and b-waves were measured at the maximal negative and positive peaks of the recordings with respect to the baseline before stimulation. The results are shown in FIG. 32.

As shown in FIG. 32, a- and b-wave amplitudes were significantly recovered in the group injected with Mito-K2.

### Related National R&D Projects

Project Unique ID: 1711170028
Project Number: 2020R1A2C3005939
Ministry: Ministry of Science and ICT
Project Management (Specialized) Institution: National Research Foundation of Korea
Research Program: Basic Research for Individuals (Ministry of Science and ICT)
Research Project Title: Intracellular Polymerization and Cell Fate Control
Contribution Rate: ½
Project Performing Institution: Ulsan National Institute of Science and Technology (UNIST)
Research Period: 2022.03.01-2023.02.28
Project Unique ID: 1711155138
Project Number: 2020M3A9D8038192
Ministry: Ministry of Science and ICT
Project Management (Specialized) Institution: National Research Foundation of Korea
Research Program: Bio & Medical Technology Development Program
Research Project Title: Development of Practical Methods for Selective Senescent Cell Therapy to Control Senescent Cell Fate
Contribution Rate: ½
Project Performing Institution: Ulsan National Institute of Science and Technology (UNIST)
Research Period: 2022.01.01-2022.12.31

1. A method for preventing or treating a retinal disease, comprising administering a compound represented by Formula 1 or a pharmaceutically acceptable salt thereof to a subject in need thereof: 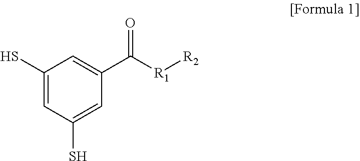
wherein, in Formula 1, R₁ is a mitochondria-targeting peptide and R₂ is a senescent cell-targeting peptide.

2. The method of claim 1, wherein the mitochondria-targeting peptide is Lys-Leu-Ala-Lys (KLAK) or Lys-Leu-Ala-Lys-Leu-Ala-Lys (KLAKLAK).

3. The method of claim 1, wherein the senescent cell-targeting peptide is Arg-Gly-Asp (RGD).

4. (canceled)

5. The method of claim 1, wherein the compound is capable of undergoing oxidation to form disulfide bonds with other molecules of the compound.

6. The method pharmaceutical composition of claim 1, wherein the compound forms a self-assembled polymer via oligomerization within mitochondria of senescent cells.

7. The method of claim 1, wherein the compound induces apoptosis in mitochondria of senescent cells.

8. The method of claim 1, wherein the retinal disease is one selected from the group consisting of macular degeneration, diabetic retinopathy, choroidal neovascularization, and retinal edema.

9. The method of claim 8, wherein the macular degeneration is one or more selected from the group consisting of age-related macular degeneration (AMD), wet macular degeneration, and dry macular degeneration.

10. (canceled)

11. (canceled)

12. (canceled)

13. (canceled)

14. (canceled)

15. The method of claim 1, comprising increasing oxygen consumption rate (OCR) of retina and RPE cells in the subject.

16. The method of claim 1, comprising restoring electroretinogram (ERG) to the level of normal cells in the subject.

17. The method of claim 1, wherein the retinal disease is caused by cells in which the expression of the Gas6, Serping1, Tmem176a, and Vim genes is increased compared to normal cells.

18. The method of claim 1, wherein the retinal disease is caused by cells in which the expression of the TOM20 protein is increased compared to normal cells.

19. A method for increasing oxygen consumption rate (OCR) of retina and RPE cells in the subject, comprising administering a compound represented by Formula 1 or a pharmaceutically acceptable salt thereof: 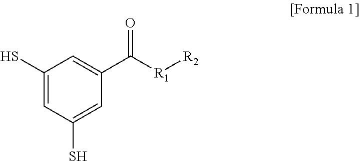
wherein, in Formula 1, R₁ is a mitochondria-targeting peptide and R₂ is a senescent cell-targeting peptide.
